(19) United States
(12) Patent Application Publication
(10) Pub. No.: US 2004/0192761 A1
(43) Pub. Date: Sep. 30, 2004

(54) Preventative and treatment effects of morinda citrifolia as an aromatase inhibitor

(76) Inventors: Afa Kehaati Palu, Orem, UT (US); Brett Justin West, Orem, UT (US); Claude Jarakae Jensen, Cedar Hills, UT (US); Chen Su, West Jordan, UT (US); Bing-Nan Zhou, Sandy, UT (US); Stephen P. Story, Alpine, UT (US)

Correspondence Address: KIRTON & McCONKIE, 1800 Eagle Gate Tower, 60 East South Temple, Salt Lake City, UT, 84111, US

(21) Appl. No.: 10/396,868

(22) Filed: Mar. 25, 2003

(51) Int. Cl. A61K 31/353; A61K 35/78

(52) U.S. Cl. 514/456; 424/769; 424/777

(57) ABSTRACT

The present invention is directed to methods of inhibiting Aromatase or Aromatase enzymes that function to convert androgens to estrogens, inhibiting receptors from binding with estrogen, and reducing and/or regulating estrogen production, as well as reducing the amount of estrogen produced within the body and regulating such production. The present invention methods and compositions effectively function to treat estrogen-dependent cancers, and particularly inhibit, destroy, and reverse the effects of of estrogen-dependent cancerous tumors through the introduction into the body (e.g. ingesting) a safe, pre-determined dosage of a naturaceutical composition formulated with or comprising one or more processed Morinda citrifolia products for a safe, pre-determined duration.

### BACKGROUND

1. Field of the Invention

The present invention relates to naturaceuticals, and particularly to a naturaceutical composition formulated as an Aromatase inhibitor, wherein the naturaceutical composition is formulated with one or more processed Morinda Citrifolia products as derived from the Indian Mulberry plant.

2. Background of the Invention and Related Art

Aromatase is part of an enzyme complex known as Cytochrome P450, which exists in different parts of the body, and particularly within several major organs. The Aromatase enzyme, in humans, is expressed in the placenta, adipose tissues, hair follicles, muscles, bones, liver and the brain. In the brain, Aromatase can be found in the anterior and mediobasal hypothalamus.

Aromatase specializes in converting C19 androgens to aromatic C18 estrogenic steroids. However, Aromatase also has the ability to metabolize xenobiotics. The ability or function of Aromatase to convert adrenal androgen substrates into estrogens accounts for the sole source of estrogen in postmenopausal women. Therefore, inhibitors of the Aromatase enzyme are used to treat postmenopausal breast cancer and other estrogen-dependent diseases.

Only recently have Aromatase inhibitors begun to be recognized as a viable treatment option for breast cancer and other estrogen-dependent diseases. As such, use of these inhibitors continues to gain momentum. Aromatase inhibitors belong to a family of hormonal treatments that have been shown in the laboratory, as well as in several clinical trials, to produce significant anti-cancerous activity in relation to breast cancer that is discovered in post-menopausal women. Aromatase inhibitors are especially advantageous or effective in cases where a woman exhibits or possesses greater estrogen-sensitivity.

In has been established and is widely believed that more than two thirds of breast cancer cases are considered “estrogen sensitive” because they are able to grow and proliferate throughout the mammary region and beyond. In response, and to prevent this overgrowth, Aromatase inhibitors are introduced, which reduce the amount of circulating estrogen in post-menopausal women, thus causing the estrogen-sensitive or estrogen dependant tumors to stop growing and even shrink. Estrogen sensitive cancers are also known as ER+(estrogen-receptor positive) and are referred to by others as progesterone-receptor positive (PR+).

Cancerous or tumor cells each have receptors (docking places) located on their cell membrane. As estrogen is produced and released into the body, it binds to these cell receptors. Therefore, in determining the efficacy of inhibitory treatments, it is possible to measure each receptor and its binding efficiency with estrogen. This binding efficiency is commonly referred to as “receptor status.” Concomitantly, receptor status becomes an invasive part of the prognosis of breast cancer.

Many Aromatase inhibitors inhibit the receptors from binding with estrogen, while others inhibit the actual Aromatase enzymes that function to convert androgens to estrogens. In addition, Aromatase inhibitors lower estrogen more effectively after menopause because in menopause, both ovaries stop producing estrogen. However, that is not to say that no amount of estrogen is produced in the body. The low level of estrogen that is produced after menopause is a result of the Aromatase enzymes converting other naturally occurring hormones into estrogen. As such, Aromatase inhibitors were developed to effectively prevent Aromatase enzymes from being used to produce estrogen. As a result, estrogen levels in the body fall, and estrogen-dependent tumors begin to, or are more likely to, shrink and digress. In contradistinction, before menopause, estrogen is mainly produced in the ovaries, with only a small amount being produced from the Aromatase process. Numerous studies have indicated that Aromatase inhibitors do not lower estrogen levels enough in premenopausal women to affect tumor growth.

Currently, there are a very limited number of Aromatase inhibitors that are actually in use and that have been approved by the Food and Drug Administration. Some of the more widely used inhibitors are Arimidex®, Aromasin®, and Femara,® which have all gone through several clinical trials. Although effective at performing their intended function of inhibiting the Aromatase enzymes, these inhibitors induce numerous undesirable side effects in the patient, thus making their treatment less popular and less desirable. Interestingly enough, these approved treatments for postmenopausal women consist of drugs that block estrogen receptors on the surface of tumor cells and are either reversible or irreversible. However, their side effects consist of, but are not limited to, the following—sweating, hot flashes, fatigue, appetite changes, headaches, bone pain, chest pain, coughing, shortness of breath, and in some patients, blood clots.

### SUMMARY AND OBJECTS OF THE INVENTION

The present invention is directed to methods of inhibiting Aromatase or Aromatase enzymes that function to convert androgens to estrogens, inhibiting receptors from binding with estrogen, and reducing and/or regulating estrogen production. as well as reducing the amount of estrogen produced within the body and regulating such production through the inhibition of Aromatase, for the treatment of cancer, and particularly for the treatment of estrogen-dependent cancerous tumors, through the introduction into the body (e.g. ingesting) a safe, pre-determined dosage of a naturaceutical composition formulated with or comprising one or more processed Morinda citrifolia products for a safe, pre-determined duration.

In one currently preferred embodiment, a quantity of a processed Morinda citrifolia product is obtained in the form of fruit juice, puree juice or juice puree, pulp, seed oil, and/or dietary fiber, using the process(es) as described below. Subsequently, an amount of any one of or a combination of these is formulated with other ingredients to create a naturaceutical composition formulated to provide significant health advantages and to assist in the treatment of and provide preventative effects upon cancerous cells through the inhibition of Aromatase enzymes.

To achieve the foregoing objects, and in accordance with the invention as embodied and broadly described herein, the present invention features a naturaceutical composition formulated with at least one processed Morinda citrifolia product for the inhibition of Aromatase or Aromatase enzymes within the body of a mammal; and a method of administering the same.

The present invention naturaceutical composition comprises at least one processed Morinda citrifolia product in one of its several forms (preferably the fruit juice), formulated with other ingredients, either natural or artificial, as needed. The preferred naturaceutical composition is a liquid that may be administered orally or through intravenous injection, wherein the active ingredients, namely Morinda citrifolia, are allowed to be absorbed into the tissues to inhibit Aromatase and reduce/regulate estrogen production.

The present invention further features a method of inhibiting Aromatase and treating, inhibiting, preventing, and reversing cancer cell growth through the prophylactic administration of a naturaceutical composition comprising at least one processed Morinda citrifolia product as an active ingredient.

### DETAILED DESCRIPTION OF THE PREFERRED EMBODIMENTS

It will be readily understood that the components of the present invention, as generally described herein, could be arranged and designed in a wide variety of different configurations. Thus, the following more detailed description of the embodiments of the system and method of the present invention is not intended to limit the scope of the invention, as claimed, but is merely representative of the presently preferred embodiments of the invention.

The present invention describes and features a method and formulation for inhibiting Aromatase and for treating and preventing cancerous cell growth within a mammal, as well as for reducing estrogen production in mammals that contributes to the growth of estrogen-dependent cancerous tumors, each through the prophylactic administration of a naturaceutical formulation comprising at least one Morinda citrifolia product in processed form.

The presently preferred embodiments of the invention will be best understood, and its benefits and advantages more clearly pointed out, by separating the following more detailed description into sections, the first pertaining to a general discussion regarding Morinda citrifolia, including its origins, processing techniques, and health benefits, as well as the methods employed to produce and manufacture the processed Morinda citrifolia products used as key active ingredients in the naturaceutical formulations described herein; and the second being a more detailed and specific discussion on the naturaceutical formulations and compositions comprising the processed Morinda citrifolia product used to inhibit Aromatase and reduce/regulate estrogen production within the body, as well as the various methods for administering these naturaceuticals to inhibit the growth, proliferation, metastasizing, and vitality of estrogen-dependent cancerous cells. Examples of experimental studies and the results obtained are also provided herein.

### GENERAL DISCUSSION OF MORINDA CITRIFOLIA AND THE METHODS USED TO PRODUCE PROCESSED MORINDA CITRIFOLIA PRODUCTS

The Indian Mulberry or Noni plant, known scientifically as Morinda Citrifolia L. (Morinda citrifolia), is a shrub or small tree up to 10 m in height. The leaves are oppositely arranged with an elliptic to ovate form. The small white flowers are contained in a fleshy, globose, head-like cluster. The fruits are large, fleshy, and ovoid. At maturity, they are creamy-white and edible, but have an unpleasant taste and odor. The plant is native to Southeast Asia and has spread in early times to a vast area from India to eastern Polynesia. It grows randomly in the wild, and it has been cultivated in plantations and small individual growing plots. The Morinda citrifolia flowers are small, white, three to five lobed, tubular, fragrant, and about 1.25 cm long. The flowers develop into compound fruits composed of many small drupes fused into an ovoid, ellipsoid or roundish, lumpy body, with waxy, white, or greenish-white or yellowish, semi-translucent skin. The fruit contains “eyes” on its surface, similar to a potato. The fruit is juicy, bitter, dull-yellow or yellowish-white, and contains numerous red-brown, hard, oblong-triangular, winged 2-celled stones, each containing four seeds.

When fully ripe, the fruit has a pronounced odor like rancid cheese. Although the fruit has been eaten by several nationalities as food, the most common use of the Morinda citrifolia plant was as a red and yellow dye source. Recently, there has been an interest in the nutritional and health benefits of the Morinda citrifolia plant, further discussed below.

Because the Morinda citrifolia fruit is for all practical purposes inedible, the fruit must be processed in order to make it palatable for human consumption and included in the naturaceutical used to inhibit Aromatase and treat various cancer cells. Processed Morinda citrifolia fruit juice can be prepared by separating seeds and peels from the juice and pulp of a ripened Morinda citrifolia fruit; filtering the pulp from the juice; and packaging the juice. Alternatively, rather than packaging the juice, the juice can be immediately included as an ingredient in another food product, frozen or pasteurized. In some embodiments, the juice and pulp can be pureed into a homogenous blend to be mixed with other ingredients. Other process include freeze drying the fruit and juice. The fruit and juice can be reconstituted during production of the final juice product. Still other processes include air drying the fruit and juices, prior to being masticated.

The present invention also contemplates the use of fruit juice and/or puree fruit juice extracted from the Morinda Citrifolia plant. In a currently preferred process of producing Morinda citrifolia fruit juice, the fruit is either hand picked or picked by mechanical equipment. The fruit can be harvested when it is at least one inch (2-3 cm) and up to 12 inches (24-36 cm) in diameter. The fruit preferably has a color ranging from a dark green through a yellow-green up to a white color, and gradations of color in between. The fruit is thoroughly cleaned after harvesting and before any processing occurs.

The fruit is allowed to ripen or age from 0 to 14 days, with most fruit being held from 2 to 3 days. The fruit is ripened or aged by being placed on equipment so it does not contact the ground. It is preferably covered with a cloth or netting material during aging, but can be aged without being covered. When ready for further processing the fruit is light in color, from a light green, light yellow, white or translucent color. The fruit is inspected for spoilage or for excessively green color and hard firmness. Spoiled and hard green fruit is separated from the acceptable fruit.

The ripened and aged fruit is preferably placed in plastic lined containers for further processing and transport. The containers of aged fruit can be held from 0 to 30 days. Most fruit containers are held for 7 to 14 days before processing. The containers can optionally be stored under refrigerated conditions prior to further processing. The fruit is unpacked from the storage containers and is processed through a manual or mechanical separator. The seeds and peel are separated from the juice and pulp.

The juice and pulp can be packaged into containers for storage and transport. Alternatively, the juice and pulp can be immediately processed into a finished juice product. The containers can be stored in refrigerated, frozen, or room temperature conditions.

The Morinda citrifolia juice and pulp are preferably blended in a homogenous blend, after which they may be mixed with other ingredients, such as flavorings, sweeteners, nutritional ingredients, botanicals, and colorings. The finished juice product is preferably heated and pasteurized at a minimum temperature of 181° F. (83° C.) or higher up to 212° F. (100° C.).

Another product manufactured is Morinda citrifolia puree and puree juice, in either concentrate or diluted form. Puree is essentially the pulp a separated from the seeds and is different than the fruit juice product described herein.

Each product is filled and sealed into a final container of plastic, glass, or another suitable material that can withstand the processing temperatures. The containers are maintained at the filling temperature or may be cooled rapidly and then placed in a shipping container. The shipping containers are preferably wrapped with a material and in a manner to maintain or control the temperature of the product in the final containers.

The juice and pulp may be further processed by separating the pulp from the juice through filtering equipment. The filtering equipment preferably consists of, but is not limited to, a centrifuge decanter, a screen filter with a size from 1 micron up to 2000 microns, more preferably less than 500 microns, a filter press, reverse osmosis filtration., and any other standard commercial filtration devices. The operating filter pressure preferably ranges from 0.1 psig up to about 1000 psig. The flow rate preferably ranges from 0.1 g.p.m. up to 1000 g.p.m., and more preferably between 5 and 50 g.p.m. The wet pulp is washed and filtered at least once and up to 10 times to remove any juice from the pulp. The wet pulp typically has a fiber content of 10 to 40 percent by weight. The wet pulp is preferably pasteurized at a temperature of 181° F. (83° C.) minimum and then packed in drums for further processing or made into a high fiber product.

The processed Morinda citrifolia product may also exist as a dietary fiber. Still further, the processed Morinda citrifolia product may also exist in oil form. The Morinda citrifolia oil typically includes a mixture of several different fatty acids as triglycerides, such as palmitic, stearic, oleic, and linoleic fatty acids, and other fatty acids present in lesser quantities. In addition, the oil preferably includes an antioxidant to inhibit spoilage of the oil. Conventional food grade antioxidants are preferably used.

The Morinda citrifolia plant is rich in natural ingredients. Those ingredients that have been discovered include, but are not limited to: from the leaves—alanine, anthraquinones, arginine, ascorbic acid, aspartic acid, calcium, beta-carotene, cysteine, cystine, glycine, glutamic acid, glycosides, histidine, iron, leucine, isoleucine, methionine, niacin, phenylalanine, phosphorus, proline, resins, riboflavin, serine, beta-sitosterol, thiamine, threonine, tryptophan, tyrosine, ursolic acid, and valine; from the flowers—acacetin-7-o-beta-d(+)-glucopyranoside, 5,7-dimethyl-apigenin-4′-o-beta-d(+)-galactopyranoside, and 6,8-dimethoxy-3-methylanthraquinone-1-o-beta-rhamnosyl-glucopyranoside; from the fruit—acetic acid, asperuloside, butanoic acid, benzoic acid, benzyl alcohol, 1-butanol, caprylic acid, decanoic acid, (E)-6-dodeceno-gamma-lactone, (Z,Z,Z)-8,11,14-eicosatrienoic acid, elaidic acid, ethyl decanoate, ethyl hexanoate, ethyl octanoate, ethyl palmitate, (Z)-6-(ethylthiomethyl) benzene, eugenol, glucose, heptanoic acid, 2-heptanone, hexanal, hexanamide, hexanedioic acid, hexanoic acid (hexoic acid), 1-hexanol, 3-hydroxy-2-butanone, lauric acid, limonene, linoleic acid, 2-methylbutanoic acid, 3-methyl-2-buten-1-ol, 3-methyl-3-buten-1-ol, methyl decanoate, methyl elaidate, methyl hexanoate, methyl 3-methylthio-propanoate, methyl octanoate, methyl oleate, methyl palmitate, 2-methylpropanoic acid, 3-methylthiopropanoic acid, myristic acid, nonanoic acid, octanoic acid (octoic acid), oleic acid, palmitic acid, potassium, scopoletin, undecanoic acid, (Z,Z)-2,5-undecadien-1-ol, and vomifol; from the roots—anthraquinones, asperuloside (rubichloric acid), damnacanthal, glycosides, morindadiol, morindine, morindone, mucilaginous matter, nor-damnacanthal, rubiadin, rubiadin monomethyl ether, resins, soranjidiol, sterols, and trihydroxymethyl anthraquinone-monomethyl ether; from the root bark—alizarin, chlororubin, glycosides (pentose, hexose), morindadiol, morindanigrine, morindine, morindone, resinous matter, rubiadin monomethyl ether, and soranjidiol; from the wood—anthragallol-2,3-dimethylether; (from the tissue culture): damnacanthal, lucidin, lucidin-3-primeveroside, and morindone-6beta-primeveroside; from the plant—alizarin, alizarin-alpha-methyl ether, anthraquinones, asperuloside, hexanoic acid, morindadiol, morindone, morindogenin, octanoic acid, and ursolic acid.

Recently, as mentioned, many health benefits have been discovered stemming from the use of products containing Morinda citrifolia. One benefit of Morinda citrifolia is found in its ability to isolate and produce Xeronine, which is a relatively small alkaloid physiologically active within the body. Xeronine occurs in practically all healthy cells of plants, animals and microorganisms. Even though Morinda citrifolia has a negligible amount of free Xeronine, it contains appreciable amounts of the precursor of Xeronine, called Proxeronine. Further, Morinda citrifolia contains the inactive form of the enzyme Proxeronase which releases Xeronine from Proxeronine. A paper entitled, “The Pharmacologically Active Ingredient of Noni” by R. M. Heinicke of the University of Hawaii, indicates that Morinda citrifolia is “the best raw material to use for the isolation of xeronine,” because of the building blocks of Proxeronine and Proxeronase. These building blocks aid in the isolation and production of Xeronine within the body. The function of the essential nutrient Xeronine is fourfold.

First, Xeronine serves to activate dormant enzymes found in the small intestines. These enzymes are critical to efficient digestion, calm nerves, and overall physical and emotional energy.

Second, Xeronine protects and keeps the shape and suppleness of protein molecules so that they may be able to pass through the cell walls and be used to form healthy tissue. Without these nutrients going into the cell, the cell cannot perform its job efficiently. Without Proxeronine to produce Xeronine our cells, and subsequently the body, suffer.

Third, Xeronine assists in enlarging the membrane pores of the cells. This enlargement allows for larger chains of peptides (amino acids or proteins) to be admitted into the cell. If these chains are not used they become waste.

Fourth, Xeronine, which is made from Proxeronine, assists in enlarging the pores to allow better absorption of nutrients.

Each tissue has cells which contain proteins which have receptor sites for the absorption of Xeronine. Certain of these proteins are the inert forms of enzymes which require absorbed Xeronine to become active. Thus Xeronine, by converting the body's procollagenase system into a specific protease, quickly and safely removes the dead tissue from skin. Other proteins become potential receptor sites for hormones after they react with Xeronine. Thus the action of Morinda citrifolia in making a person feel well is probably caused by Xeronine converting certain brain receptor proteins into active sites for the absorption of the endorphin, the well being hormones. Other proteins form pores through membranes in the intestines, the blood vessels and other body organs. Absorbing Xeronine on these proteins changes the shape of the pores and thus affects the passage of molecules through the membranes.

Because of its many benefits, Morinda citrifolia has been known to provide a number of anecdotal effects in individuals having cancer, arthritis, headaches, indigestion, malignancies, broken bones, high blood pressure, diabetes, pain, infection, asthma, toothaches, blemishes, immune system failure, and others.

The compositions containing Morinda citrifolia may be in a form suitable for oral use, systemic administration, injection, and others. In regards to an oral composition, such a composition may exist, for example, as tablets, or lozenges, aqueous or oily suspensions, dispersible powders or granules, emulsions, syrups or elixirs. Compositions intended for oral use may be prepared according to any method known in the art for the manufacture of Morinda citrifolia compositions and such compositions may contain one or more agents selected from the group consisting of sweetening agents, flavoring agents, coloring agents and preserving agents. Tablets contain Morinda citrifolia in admixture with non-toxic pharmaceutically acceptable excipients which are suitable for the manufacture of tablets. These excipients may be for example, inert diluents, granulating and disintegrating agents, binding agents, and lubricating agents. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed.

Aqueous suspensions contain the Morinda citrifolia in admixture with excipients suitable for the manufacture of aqueous suspensions. Such excipients are suspending agents, for example, sodium carboxymethyl-cellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitor monooleate, or condensation products of ethylene oxide with partial esters derived from fatty acids and hexitol anhydrides, for example polyethylene sorbitan monooleate.

Favorably, this invention provides a method of inhibiting Aromatase with a Morinda citrifolia-based naturaceutical formulation without any significant tendency to cause gastric side effects.

### MORINDA CITRIFOLIA-BASED NATURACEUTICAL FORMULATIONS AND METHODS OF ADMINISTRATION FOR INHIBITING CANCER CELL GROWTH WITHIN THE REGION OF THE COLON

The present invention advances Aromatase inhibitors by providing a naturaceutical composition or Aromatase inhibitor or estrogen-dependent cancer cell treatment formulated with Morinda citrifolia from the Indian Mulberry plant. The Morinda citrifolia is incorporated into various carriers or naturaceutical compositions suitable for in vivo treatment of a patient. For instance, the inhibitor may be ingested, injected, introduced intravenously, or otherwise internalized as is appropriate and directed.

In one exemplary embodiment, the naturaceutical composition of the present invention comprises one or more of a processed Morinda citrifolia product present in an amount by weight between about 0.01 and 100 percent by weight, and preferably between 0.01 and 95 percent by weight. Several embodiment of formulations are provided below. However, these are only intended to be exemplary as one ordinarily skilled in the art will recognize other formulations or compositions comprising the processed Morinda citrifolia product.

The processed Morinda citrifolia product comprises at least one of the active ingredients in the naturaceutical, or contains one or more active ingredients, such as Quercetin and Rutin, and others, for effectuating the inhibition of Aromatase and reducing and regulating estrogen production, inhibiting estrogen receptors from binding with estrogen, as well as for effectuating the destruction of estrogen-dependent cancerous cells, particularly early stage estrogen-dependent cancerous cells.

Active ingredients within the processed Morinda citrifolia product may be extracted out using various alcohol or alcohol-based solutions, such as methanol, ethanol, and ethyl acetate, and other alcohol-based derivatives using any known process in the art. The active ingredients of Quercetin and Rutin are present in amounts by weight ranging from 0.01-10 percent of the total formulation or composition. These amounts may be concentrated as well into a more potent concentration in which they are present in amounts ranging from 10 to 100 percent. The processed Morinda citrifolia product may be formulated with various other ingredients to produce various compositions, such as a naturaceutical composition, a topical dermal composition, or others. The ingredients to be utilized in a naturaceutical composition are any that are safe for introduction into the body of a mammal, and particularly a human, and may exist in various forms, such as liquids, tablets, lozenges, aqueous or oily solutions, dispersible powders or granules, emulsions, syrups, elixirs, etc. Moreover, since the naturaceutical composition is preferably consumed orally, it may contain one or more agents selected from the group consisting of sweetening agents, flavoring agents, coloring agents, preserving agents, and other medicinal agents as directed.

The ingredients to be utilized in a topical dermal composition are also any that are safe for internalizing into the body of a mammal and may exist in various forms, such as gels, lotions, creams, ointments, etc., each comprising one or more carrier agents. The ingredients for systemically (e.g. intravenously) administered formulations may also comprise any known in the art.

The present invention further features a method of administering a naturaceutical composition to a mammal to inhibit Aromatase, as well as to reduce and/or regulate estrogen production. In one exemplary embodiment, the method comprises the steps of (a) formulating a naturaceutical composition comprising in part a processed Morinda citrifolia product present in an amount between about 0.01 and 95 percent by weight, wherein the composition also comprises a carrier, such as water or purified water, and may also comprise other natural or artificial ingredients; (b) administering the naturaceutical composition into the body of a mammal, such that the processed Morinda citrifolia product is sufficiently internalized; (c) repeating the above steps as often as necessary to provide an effective amount of the processed Morinda citrifolia product to inhibit Aromatase enzymes that function to convert androgens to estrogens, inhibit receptors from binding with estrogen, and/or reduce and/or regulate estrogen production.

The step of administering the naturaceutical composition into the body preferably comprises ingesting the composition orally through one of several means. Specifically, the naturaceutical composition may be formulated as a liquid, gel, solid, or some other type that would allow the composition to be quickly digested and concentrated within the colon. It is important to note that the step of administering the naturaceutical composition should be carried out in an effective manner so that the greatest concentration of naturaceutical composition is allowed to absorb into the tissues and cells. For the naturaceutical composition to take effect, it must be sufficiently internalized. Once sufficiently internalized, it may then begin to act by inhibiting Aromatase, discouraging the binding of estrogen to receptors, and reducing the amount of estrogen produced.

In another embodiment, the step of administering the naturaceutical composition may include injecting the composition into the body using an intravenous pump. This technique is advantageous as it would allow the composition to be localized in the area where it would have the most effect, or the area that would provide for the greatest concentration of the naturaceutical composition, such as in the mammary region of a breast cancer patient.

In one exemplary embodiment, the naturaceutical composition is administered by taking between 1 teaspoon and 2 oz., and preferably 2 oz., of the naturaceutical composition every two hours each day, or at least twice a day on a continued basis. Also, the naturaceutical composition is to be taken on an empty stomach, meaning at a period of time at least two hours prior to consumption of any food or drink. Following this, the naturaceutical composition is allowed to actively inhibit Aromatase, thus positively impacting estrogen-dependent cancerous cells within the body and inhibiting their growth. Of course, one ordinarily skilled in the art will recognize that the amount of composition and frequency of use may vary from individual to individual.

The following tables illustrate or represent some of the preferred formulations or compositions contemplated by the present invention. As stated, these are only intended as exemplary embodiments and are not to be construed as limiting in any way.

| | |
|---|---|
| Ingredients | Percent by Weight |

Formulation One

| | |
|---|---|
| Morinda citrifolia puree juice or fruit juice | 100% |

Formulation Two

| | |
|---|---|
| Morinda citrifolia fruit juice | 85-99.99% |
| Water | 0.1-15% |

Formulation Three

| | |
|---|---|
| Morinda citrifolia fruit juice | 85-99.99% |
| non-Morinda citrifolia-based fruit juices | 0.1-15% |

Formulation Four

| | |
|---|---|
| Morinda citrifolia fruit juice | 50-90% |
| water | 0.1-50% |
| non-Morinda citrifolia-based fruit juices | 0.1-30% |

Formulation Five

| | |
|---|---|
| Morinda citrifolia puree juice | 85-99.9% |
| water | 0.1-15% |

Formulation Six

| | |
|---|---|
| Morinda citrifolia puree juice | 85-99.9% |
| non-Morinda citrifolia-based fruit juices | 0.1-15% |

Formulation Seven

| | |
|---|---|
| Morinda citrifolia puree juice | 50-90% |
| water | 0.1-50% |
| non-Morinda citrifolia-based fruit juices | 0.1-30% |

Formulation Eight

| | |
|---|---|
| Morinda citrifolia dietary fiber | 0.1-30% |
| water | 1-99.9% |
| non-Morinda citrifolia-based fruit juices | 1-99.9% |

Formulation Nine

| | |
|---|---|
| Morinda citrifolia dietary fiber | 0.1-30% |
| water | 1-99.9% |
| Morinda citrifolia fruit juice or puree juice | 1-99.9% |

Formulation Ten

| | |
|---|---|
| Morinda citrifolia oil | 0.1-30% |
| carrier medium | 70-99.9% |
| other ingredients | 1-95% |

Formulation Eleven

| | |
|---|---|
| Morinda citrifolia product | 10-80% |
| carrier medium | 20-90% |

Formulation Twelve

| | |
|---|---|
| Morinda citrifolia product | 5-80% |
| carrier medium | 20-95% |

Formulation Thirteen

| | |
|---|---|
| Morinda citrifolia oil or oil extract | 0.1-20% |
| carrier medium | 20-90% |

Formulation Fourteen

| | |
|---|---|
| Morinda citrifolia puree juice or fruit Juice | 0.1-80% |
| Morinda citrifolia oil | 0.1-20% |
| carrier medium | 20-90% |

Formulation Fifteen

| | |
|---|---|
| Morinda citrifolia puree juice concentrate or fruit | 100% |
| juice concentrate | |

Formulation Sixteen

| | |
|---|---|
| Morinda citrifolia fruit juice concentrate or puree | 85-99.99% |
| juice concentrate | |

Water 0.1-15%

As stated, in one exemplary embodiment, the present invention features a method for introducing an internal composition or formulation to inhibit Aromatase, reduce/regulate estrogen production, inhibit binding of estrogen to receptors, and treat, prevent, inhibit, destroy, and reverse the effects of estrogen-dependent tumors and cancer cell growth within a cancerous region, such as the mammary region. This method essentially comprises the introduction of an internal composition into the body of a mammal infected with cancerous cells. Several embodiments of the internal composition comprising various different ingredients are contemplated for use herein, with each embodiment comprising one or more forms of a processed Morinda citrifolia product as taught and explained herein and a carrier agent or medium.

In one preferred method, Aromatase or Aromatase enzymes are inhibited, and estrogen-dependent cancer and its growth is treated, prevented, destroyed, and/or reversed, by administering at least one (1) ounce of one of Formulations One through Sixteen above in the morning on an empty stomach, and at least one (1) ounce at night on an empty stomach, just prior to retiring to bed. In one example, which is not meant to be limiting in any way, the beneficial Morinda Citrifolia is processed into Tahitian Noni® juice manufactured by Morinda, Incorporated of Orem, Utah.

In one exemplary embodiment, the internal composition comprises the ingredients of: a processed Morinda citrifolia product present in an amount by weight between about 10-80 percent; and a carrier medium present in an amount by weight between about 20-90 percent.

In this embodiment, the processed Morinda citrifolia product may comprise one or more of a processed Morinda citrifolia fruit juice, processed Morinda citrifolia puree juice, processed Morinda citrifolia dietary fiber, and/or processed Morinda citrifolia oil extract product.

In another exemplary embodiment, the internal composition comprises the ingredients of: processed Morinda citrifolia fruit juice or puree juice present in an amount by weight between about 0.1-80 percent; processed Morinda citrifolia oil present in an amount by weight between about 0.1-20 percent; and a carrier medium present in an amount by weight between about 20-90 percent. Morinda citrifolia puree juice or fruit juice may also be formulated with a processed Morinda citrifolia dietary fiber product present in similar concentrations.

According to the present invention, the particular methods of introducing an internal composition may comprises any method of actually introducing the internal composition into the body of a mammal for the purposes identified herein. Although the particular methods are many, the present invention recognizes that the internal composition may be introduced intravenously, transdermally, orally, or systemically. No matter what method is employed, it is important to thoroughly internalize the composition so that the internal composition, and particularly the Morinda citrifolia and other active ingredients, can effectively inhibit Aromatase and treat any estrogen-dependent cancer, thus contributing to the abatement and subsequent inhibition and prevention of such cancer, and also so that any early stage estrogen-dependent cancerous cells can be destroyed.

The carrier medium identified in the above Formulations may comprise any ingredient capable of being introduced into the body of a mammal, and that is also capable of providing the carrying medium to the processed Morinda citrifolia product. Specific carrier mediums formulations are well known in the art and not described in detail herein. The purpose of the carrier medium is as stated, to provide a means to embody the processed Morinda citrifolia product within the internal composition that is capable of being introduced into the body.

The following examples set forth and present the preventative and treatment effects of the processed Morinda citrifolia products on Aromatase and estrogen-dependent cancerous cells, as well as the preventative and treatment effects of Morinda citrifolia against the proliferation or metastasizing of these cancerous cells. These examples are not intended to be limiting in any way, but are merely illustrative of the benefits and advantageous, as well as the remedial effects, of the Morinda citrifolia products.

### Example One

Current prior art treatments available for postmenopausal women with breast cancer comprise treatments for either blocking or inhibiting estrogen receptors or blocking Aromatase activity. However, these treatments are unable to accomplish both. Recent experiments were conducted using in-vitro bioassays to evaluate the Aromatase enzyme inhibiting and radioligand binding activities of Morinda citrifolia, in the form of Tahitian Noni® Puree Juice.

In this experiment, human recombinant cells were used as the sources of estrogen receptors ER_{α} and ER_{β}. Each of the positive control enzymes, Diethylstilbestrol Aromatase, and CYP450 2C19, were of human origin.

Tahitian Noni® puree juice having a concentration of 1% was first investigated. The results of this test indicated that the active ingredient of Morinda citrifolia caused or induced a 89% Aromatase inhibition with the CYP450 2C19 enzyme receptor, a 99% Aromatase inhibition of the estrogen-alpha enzyme receptors, and a 102% Aromatase inhibition of the estrogen-beta enzyme receptors. These results seems to suggest that Morinda citrifolia puree juice at the concentration used had a significant impact on the inhibition of Aromatase and estrogen receptors, which inhibition compares favorably to current prior art treatments.

Furthermore, it was found that Morinda citrifolia inhibited 81% of the 1A2, 89% of the 2C19, 81% of the 2C9, 93% of the 2D6, and 77% of the 3A4 CYP450 enzymes.

The present invention may be embodied in other specific forms without departing from its spirit of essential characteristics. The described embodiments are to be considered in all respects only al illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A method for inhibiting Aromatase and Aromatase enzymes that function to convert androgens to estrogens, said method comprising introducing into a mammal a composition comprising a processed Morinda citrifolia product present in an amount between about 0.01 and 100 percent by weight, said processed Morinda citrifolia product comprising the active ingredient Quercetin.

2. The method of claim 1, wherein said Quercetin is present in an amount between about 0.1 and 10 percent by weight.

3. The method of claim 1, wherein said processed Morinda citrifolia product is Morinda citrifolia fruit juice.

4. The method of claim 1, wherein said processed Morinda citrifolia product is Morinda citrifolia oil extract.

5. The method of claim 1, wherein said processed Morinda citrifolia product is Morinda citrifolia dietary fiber.

6. The method of claim 1, wherein said processed Morinda citrifolia product is Morinda citrifolia puree juice.

7. The method of claim 1, wherein said processed Morinda citrifolia product is Morinda citrifolia puree.

8. The method of claim 1, wherein said processed Morinda citrifolia product further comprises Rutin as an additional active ingredient that synergistically works with said Quercetin to inhibit said Aromatase.

9. The method of claim 8, wherein said Rutin is present in an amount between about 0.1 and 10 percent by weight.

10. The method of claim 1, wherein said composition is administered orally.

11. The method of claim 1, wherein said composition is administered transdermally to said infected area.

12. The method of claim 1, wherein said composition is administered by injection into said infected area.

13. The method of claim 1, wherein said composition is administered intravenously.

14. The method of claim 1, wherein said composition is administered systemically.

15. The method of claim 1, wherein said processed Morinda citrifolia product comprises processed Morinda citrifolia fruit juice concentrate.

16. The method of claim 1, wherein said processed Morinda citrifolia product comprises processed Morinda citrifolia puree juice concentrate.

17. A method for inhibiting Aromatase and preventing the formation and growth of estrogen-dependent cancerous cells within a mammal, said method comprising the steps of:
adding a processed Morinda citrifolia product to an alcohol-based solution;
isolating and extracting an active ingredient of said processed Morinda citrifolia product from said solution;
introducing said extracted active ingredient into said mammal, wherein said extracted active ingredient inhibits, prevents, destroys, and reverses the effects of said estrogen-dependent cancerous cells.

18. The method of claim 17, wherein said processed Morinda citrifolia product comprises processed Morinda citrifolia fruit juice.

19. The method of claim 17, wherein said processed Morinda citrifolia product comprises processed Morinda citrifolia puree.

20. The method of claim 17, wherein said alcohol-based solution is selected from the group consisting essentially of methanol, ethanol, and ethyl acetate, and other alcohol-based derivatives.

21. The method of claim 17, wherein said active ingredient is Quercetin.

22. The method of claim 21, wherein said active ingredient is Rutin that synergistically works with said Quercetin to inhibit said Aromatase and prevent said formation and growth of said estrogen-dependent cancerous cells.

23. The method of claim 17, wherein said step of introducing said extracted active ingredient comprises orally administering two ounces of a food product to a patient twice daily each day on an empty stomach, said food product comprising said active ingredient obtained from said processed Morinda citrifolia product.

24. The method of claim 17, wherein said step of introducing said extracted active ingredient comprises applying a topical composition to said area, said topical composition comprising said active ingredient obtained from said processed Morinda citrifolia product.

25. A method for inhibiting Aromatase and preventing the formation and growth of estrogen-dependent cancerous cells within a mammal, said method comprising the steps of:
orally administering to said mammal at least one ounce of a naturaceutical formulation comprising a processed Morinda citrifolia product on an empty stomach in the morning;
orally administering to said mammal at least one ounce of said naturaceutical formulation on an empty stomach prior to sleeping at night; and
repeating said steps of orally administering each day until said estrogen-dependent cancerous cells are destroyed.

26. The method of claim 25, wherein said naturaceutical formulation comprises:
processed Morinda citrifolia fruit juice present in an amount by weight of about 100 percent.

27. The method of claim 25, wherein said naturaceutical formulation comprises:
processed Morinda citrifolia fruit juice present in an amount by weight between about 85-99.99 percent; and
water present in an amount by weight between about 0.1-15 percent.

28. The method of claim 25, wherein said naturaceutical formulation comprises:
processed Morinda citrifolia fruit juice present in an amount by weight between about 85-99.99 percent; and
non-Morinda citrifolia-based fruit juices present in an amount by weight between about 0.1-15 percent.

29. The method of claim 25, wherein said naturaceutical formulation comprises:
processed Morinda citrifolia fruit juice present in an amount by weight between about 50-90 percent;
water present in an amount by weight between about 0.1-50 percent; and
non-Morinda citrifolia-based fruit juices present in an amount between about 0.1-30 percent.

30. The method of claim 25, wherein said naturaceutical formulation comprises:
processed Morinda citrifolia puree juice present in an amount by weight of about 100 percent.

31. The method of claim 25, wherein said naturaceutical formulation comprises:
processed Morinda citrifolia puree juice present in an amount by weight between about 85-99.99 percent; and
water present in an amount by weight between about 0.1-15 percent.

32. The method of claim 25, wherein said naturaceutical formulation comprises:
processed Morinda citrifolia puree juice present in an amount by weight between about 85-99.99 percent; and
non-Morinda citrifolia-based fruit juices present in an amount by weight between about 0.1-15 percent.

33. The method of claim 25, wherein said naturaceutical formulation comprises:
processed Morinda citrifolia puree juice present in an amount by weight between about 50-90 percent;
water present in an amount by weight between about 0.1-50 percent; and
non-Morinda citrifolia-based fruit juices present in an amount between about 0.1-30 percent.

34. The method of claim 25, wherein said step of orally administering comprises orally administering two ounces of said naturaceutical formulation.

35. A method for regulating estrogen production and inhibiting the ability of estrogen receptors from binding with estrogen, said method comprising the steps of:
orally administering at least one ounce of a naturaceutical formulation comprising
a processed Morinda citrifolia product in the morning on an empty stomach; and
orally administering at least one ounce of said naturaceutical formulation prior to sleeping at night.

36. The method of claim 35, wherein said naturaceutical formulation comprises:
processed Morinda citrifolia fruit juice present in an amount by weight between about 100 percent.

37. The method of claim 35, wherein said naturaceutical formulation comprises:
processed Morinda citrifolia fruit juice present in an amount by weight between about 85-99.99 percent; and
water present in an amount by weight between about 0.1-15 percent.

38. The method of claim 35, wherein said naturaceutical formulation comprises:
processed Morinda citrifolia fruit juice present in an amount by weight between about 85-99.99 percent; and
non-Morinda citrifolia-based fruit juices present in an amount by weight between about 0.1-15 percent.

39. The method of claim 35, wherein said naturaceutical formulation comprises:
processed Morinda citrifolia fruit juice present in an amount by weight between about 50-90 percent;
water present in an amount by weight between about 0.1-50 percent; and
non-Morinda citrifolia-based fruit juices present in an amount between about 0.1-30 percent.

40. The method of claim 35, wherein said naturaceutical formulation comprises:
processed Morinda citrifolia puree juice present in an amount by weight of about 100 percent.

41. The method of claim 35, wherein said naturaceutical formulation comprises:
processed Morinda citrifolia puree juice present in an amount by weight between about 85-99.99 percent; and
water present in an amount by weight between about 0.1-15 percent.

42. The method of claim 35, wherein said naturaceutical formulation comprises:
processed Morinda citrifolia puree juice present in an amount by weight between about 85-99.99 percent; and
non-Morinda citrifolia-based fruit juices present in an amount by weight between about 0.1-15 percent.

43. The method of claim 35, wherein said naturaceutical formulation comprises:
processed Morinda citrifolia puree juice present in an amount by weight between about 50-90 percent;
water present in an amount by weight between about 0.1-50 percent; and
non-Morinda citrifolia-based fruit juices present in an amount between about 0.1-30 percent.

44. The method of claim 35, wherein said step of orally administering comprises orally administering two ounces of said naturaceutical formulation twice daily each day.

45. A method for inhibiting Aromatase and the ability of estrogen to bind with estrogen receptors to treat, prevent, inhibit, destroy, and reverse the effects of estrogen-dependent cancerous cells within a mammal, said method comprising the steps of:
introducing, systemically, an internal composition into said mammal, said internal composition comprising:
a processed Morinda citrifolia product present in an amount by weight between about 10-80 percent; and
a carrier medium present in an amount by weight between about 20-90 percent.

46. The method of claim 45, wherein said processed Morinda citrifolia product comprises processed Morinda citrifolia fruit juice.

47. The method of claim 45, wherein said processed Morinda citrifolia product comprises processed Morinda citrifolia puree juice.

48. The method of claim 45, wherein said processed Morinda citrifolia product comprises processed Morinda citrifolia dietary fiber.

49. The method of claim 45, wherein said step of introducing an internal composition selected from the group consisting of intravenous introduction, oral introduction, and injection.

50. A method for inhibiting Aromatase and regulating estrogen production, said method comprising the steps of:
introducing an internal composition to a mammal, said internal composition comprising:
a processed Morinda citrifolia product present in an amount by weight between about 5-80 percent; and
a carrier medium present in an amount by weight between about 20-95 percent.

51. The method of claim 50, wherein said processed Morinda citrifolia product is selected from the group consisting of processed Morinda citrifolia fruit juice, processed Morinda citrifolia puree juice, processed Morinda citrifolia dietary fiber, processed Morinda citrifolia oil, and processed Morinda citrifolia oil extract.

52. The method of claim 50, wherein said step of introducing an internal composition comprises methods selected from the group consisting of intravenous introduction, oral introduction, transdermal introduction, and injection.

53. An Aromatase inhibitor for treating estrogen-dependent tumorous cells in mammals comprising:
a naturaceutical composition comprising at least one processed Morinda citrifolia product;

54. The Aromatase inhibitor of claim 53, wherein said processed Morinda citrifolia product comprises Morinda citrifolia fruit juice.

55. The Aromatase inhibitor of claim 53, wherein said processed Morinda citrifolia product comprises Morinda citrifolia puree juice.

56. The Aromatase inhibitor of claim 53, wherein said processed Morinda citrifolia product comprises Morinda citrifolia puree juice concentrate.

57. The formulation of claim 53, wherein said processed Morinda citrifolia product comprises Morinda citrifolia fruit juice concentrate.

58. The formulation of claim 53, wherein said processed Morinda citrifolia product comprises Morinda citrifolia dietary fiber.

59. The formulation of claim 53, wherein said processed Morinda citrifolia product of said naturaceutical formulation further comprises an active ingredient Quercetin present in an amount between about 0.1 and 10 percent by weight.

60. The formulation of claim 53, wherein said processed Morinda citrifolia product is present in an amount between about 0.01 and 100 percent by weight.

61. The formulation of claim 59, wherein said processed Morinda citrifolia product further comprises Rutin as an additional active ingredient that synergistically works with said Quercetin to treat said migraine headaches and its associated symptoms.

62. The formulation of claim 61, wherein said Rutin is present in an amount between about 0.1 and 10 percent by weight.

63. The formulation of claim 53, wherein said naturaceutical is administered orally.

64. The formulation of claim 53, wherein said naturaceutical is administered transdermally to said infected area.

65. The formulation of claim 53, wherein said naturaceutical is administered intravenously.

66. The formulation of claim 53, wherein said naturaceutical is administered systemically.

67. A method for regulating the production of estrogen within the body of a mammal for the treatment of estrogen-dependent tumors, said method comprising the steps of:
introducing, systemically, an internal composition into said mammal, said internal composition comprising:
a processed Morinda citrifolia product present in an amount by weight between about 0.1 and 100 percent.
